(19) United States
(12) Patent Application Publication
(10) Pub. No.: US 2009/0107876 A1
(43) Pub. Date: Apr. 30, 2009

(54) Post-surgical accessory convenience kit

(76) Inventor: Bradley P. Bengtson, Grand Rapids, MI (US)

Correspondence Address: RYAN KROMHOLZ & MANION, S.C., POST OFFICE BOX 26618, MILWAUKEE, WI, 53226, US

(21) Appl. No.: 12/288,255

(22) Filed: Oct. 17, 2008

Related U.S. Application Data
(60) Provisional application No. 60/999,328, filed on Oct. 17, 2007.

(51) Int. Cl. A61M 27/00; A61B 19/02; A45C 13/30; A45C 5/14

(52) U.S. Cl. 206/570; 604/543; 190/115; 190/18 A

(57) ABSTRACT

A post-surgical accessory convenience kit, or PACK includes the plurality of items, which serve a patient's post-operative needs, such as, but not limited to, a drain suction bulb (reservoir) and attendant tubing; dressing change materials; post-operative medication (e.g., pain medication, antibiotics, ointments, and other medication prescribed by a physician); physician or caregiver instructions, drain record sheets, and caregiver contact phone numbers. The kit further includes a carrier or pouch. The pouch includes a plurality of pockets or compartments that are sized and configured to hold the diverse array of items, which are directly related to the patient's post-operative needs. The kit can be worn about the waist or shoulder of an individual, and can add post-operative comfort by taking tension off of an incision and drain entry site and avoiding accidental pulling of the drain or suction bulb.

### RELATED APPLICATIONS

This application claims the benefit of co-pending U.S. Provisional Patent Application Ser. No. 60/999,328, filed 17 Oct. 2007.

### FIELD OF THE INVENTION

This application relates generally to the drainage of fluid from the body during the wound healing process, e.g., following surgery, trauma, or placement of implants or surgical devices, and, more particularly, to a post-surgical, post-operative kit sized to be worn by a patient to carry items that support their post-surgical needs.

### BACKGROUND OF THE INVENTION

Surgical drains are utilized in approximately 50% of major surgical procedures. Drains are required in many patients because the body cannot absorb the extra blood and fluid that occurs following many surgical procedures. A representative prior art continuous drain system can comprise an implanted device such as a piece of rubber tubing (Penrose drain) (as shown in FIG. 1), which provides dependent gravity drainage or responds to a negative suction force generated by a manual closed suction bulb. These types of drains constitute the most common devices currently available.

Drains, when they are used, are typically painful at the point they exit the skin, and can easily pull at these exit points. The suction bulbs that collect the fluid are typically attached to clothing with a safety pin or placed in a pocket, but commonly the tubing or bulb can get caught or pull at the skin or even be pulled out accidentally. As a result, patients do not like drains.

There is a need for a post-surgical, post-operative pack, not for any instruments, but for those items necessary for a patient's comfort, convenience, and post-surgical needs.

### SUMMARY OF THE INVENTION

The invention provides a post-surgical accessory convenience kit (in shorthand PACK). The kit is well suited for, but not limited to, serving the needs of patients who require drains.

One aspect of the invention provides a kit including a reservoir, a length of tubing, and a pouch. The length of tubing has a first end adapted to be coupled to the reservoir and a second end adapted to be implanted into a drain site in a patent.

An additional aspect of the invention is a kit including dressing change materials.

An additional aspect of the invention is a kit including post-operative medication. The post-operative medication includes at least one of the following: pain medication, antibiotics, and/or ointments.

An additional aspect of the invention is a kit including at least one set of post-operative instructions.

An additional aspect of the invention is a kit including at least one drain record.

An additional aspect of the invention is a kit including at least one physician contact phone number.

An additional aspect of the invention is a kit including a pouch that is made of a material that is water and fluid resistant.

An additional aspect of the invention is a kit including a pouch made of a material that is clear.

An additional aspect of the invention is a kit including a pouch including a strap. In one embodiment the strap is sized and configured to secure the pouch around the waist of a patient. In an alternative embodiment the strap is sized and configured to wear the pouch over the shoulder of a patient.

An additional aspect of the invention is a kit including a pouch containing at least one pocket. An additional aspect of the invention is a kit wherein at least one of the pockets on the pouch includes a closure element.

An additional aspect of the invention is a kit including at least one auxiliary case attached to the outside of the pouch.

An additional aspect of the invention is a method for providing post-operative materials to a patient. The method may include providing a pouch with a plurality of compartments, providing a reservoir, providing tubing, providing post-operative medications, providing post-operative information, providing dressing change materials. The method may further include arranging the reservoir, tubing, post-operative medications, post-operative information, and dressing change materials within the compartments of the pouch. The method may further include distributing the pouch to a patient.

An additional aspect of the invention is a kit including a reservoir, a length of tubing, dressing change materials, post-operative medication, post-operative care instructions, physician contact phone numbers; and drain records. The length of tubing may have a first end adapted for coupling to the reservoir and a second end adapted for implanting into a drain site in a patient.

An additional aspect of the invention is a kit including a pouch, the pouch having a plurality of compartment.

An additional aspect of the invention is a kit wherein each of the items, including the reservoir, dressing change materials, post-operative medication, post-operative care instructions, physician contact phone numbers, and drain records are contained within one of the compartments of the pouch.

An additional aspect of the invention is a luggage article having a frame with an interior volume, a handle coupled to the frame, and material carried by the frame to enclose the interior volume. The material may be transparent such that substantially the entire interior volume is visible from outside the frame.

An additional aspect of the invention is a luggage article with a telescoping handle.

An additional aspect of the invention is a luggage article with least one wheel coupled to the frame.

An additional aspect of the invention is a luggage article having a body having an interior volume, the body being made of a transparent material such that substantially the entire interior volume is visible from outside the body. The body may have an opening to provide access to the interior volume and a handle and/or strap coupled to the body.

An additional aspect of the invention is a luggage article wherein the interior volume is sized and configured to receive a laptop computer.

### BRIEF DESCRIPTION OF THE DRAWINGS

FIG. 1 is an anatomic side section prior art view of a human abdomen showing an interior wound area and a tube that is placed according to conventional techniques to drain fluid from a seroma at the wound site.

FIG. 2 is a view showing a representative collection of the plurality of items that a patient having a post-operative drain desires or requires for their post-operative needs.

FIG. 3 shows a post-surgical accessory convenience kit, or PACK that includes the plurality of items shown in FIG. 2, as well as a carrier or pouch that includes a plurality of pockets or compartments that are sized and configured to hold the diverse array of items shown in FIG. 2.

FIGS. 4 and 5 show the placement of the items shown in FIG. 2 within the pouch shown in FIG. 3.

FIG. 6 shows the kit shown in FIG. 3 when in use, being worn about the waist of a patient.

FIG. 7 shows the kit shown in FIG. 3 when in use, being worn about the shoulder of a patient.

FIG. 8 is a front elevation view of an alternative embodiment of a post-surgical accessory convenience kit.

FIG. 9 is a rear elevation view of an alternative embodiment of the post-surgical accessory convenience kit of FIG. 8.

FIG. 10 is a left side elevation view of an alternative embodiment of a post-surgical accessory convenience kit of FIG. 8.

FIG. 11 is a right side elevation view of an alternative embodiment of a post-surgical accessory convenience kit of FIG. 8.

FIG. 12 is a top plan view of an alternative embodiment of a post-surgical accessory convenience kit of FIG. 8.

FIG. 13 is a bottom plan view of an alternative embodiment of a post-surgical accessory convenience kit of FIG. 8.

FIG. 14 is a perspective view of an embodiment of a see-through computer bag.

FIG. 15 is an embodiment of a see-through carry on bag.

FIG. 16 is an embodiment of a see-through suitcase.

### DESCRIPTION OF REPRESENTATIVE EMBODIMENTS

Although the disclosure hereof is detailed and exact to enable those skilled in the art to practice the invention, the physical embodiments herein disclosed merely exemplify the invention that may be embodied in other specific structure. While the preferred embodiment has been described, the details may be changed without departing from the invention, which is defined by the claims.

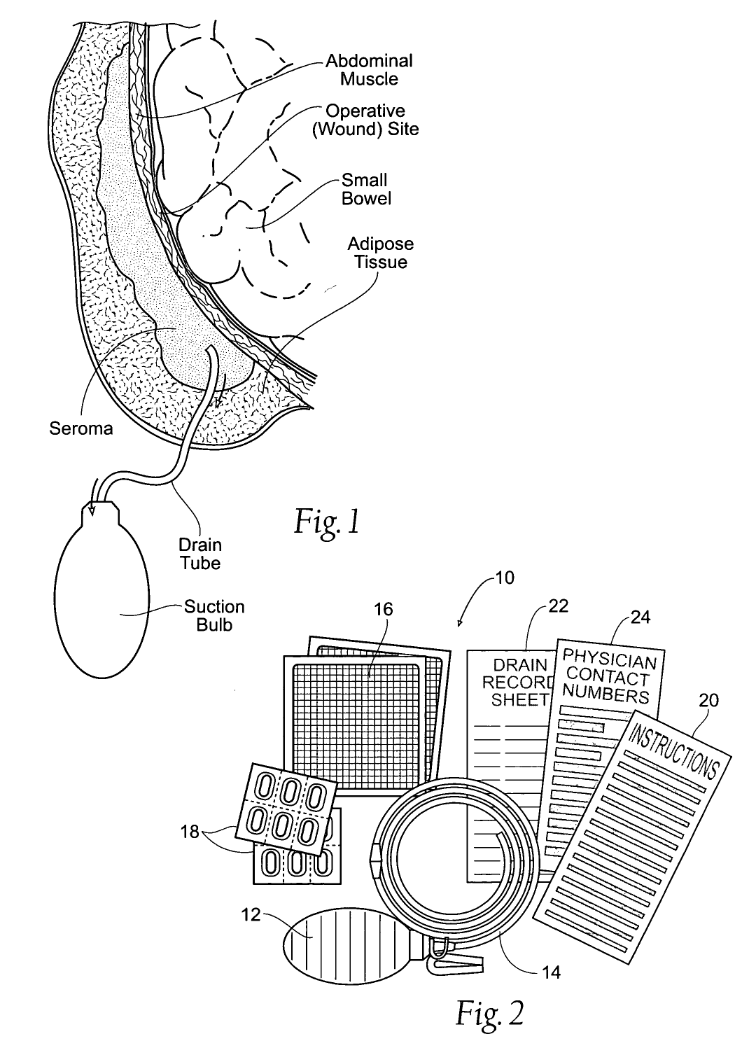

FIG. 2 shows a representative collection of the plurality of items 10 that a patient having a post-operative drain desires or requires for their post-operative needs. As shown in FIG. 2, the items 10 include, but not limited to, a drain suction bulb 12 (reservoir) and attendant tubing 14 (which, in use, is implanted at a drain site (as shown, e.g., in FIG. 1). The items 10 can further include dressing change materials 16, and post-operative medication 18 (e.g., pain medication, antibiotics, ointments, and other medication prescribed by a physician). The items 10 can further include physician or caregiver instructions 20, drain record sheets 22, and caregiver contact phone numbers 24.

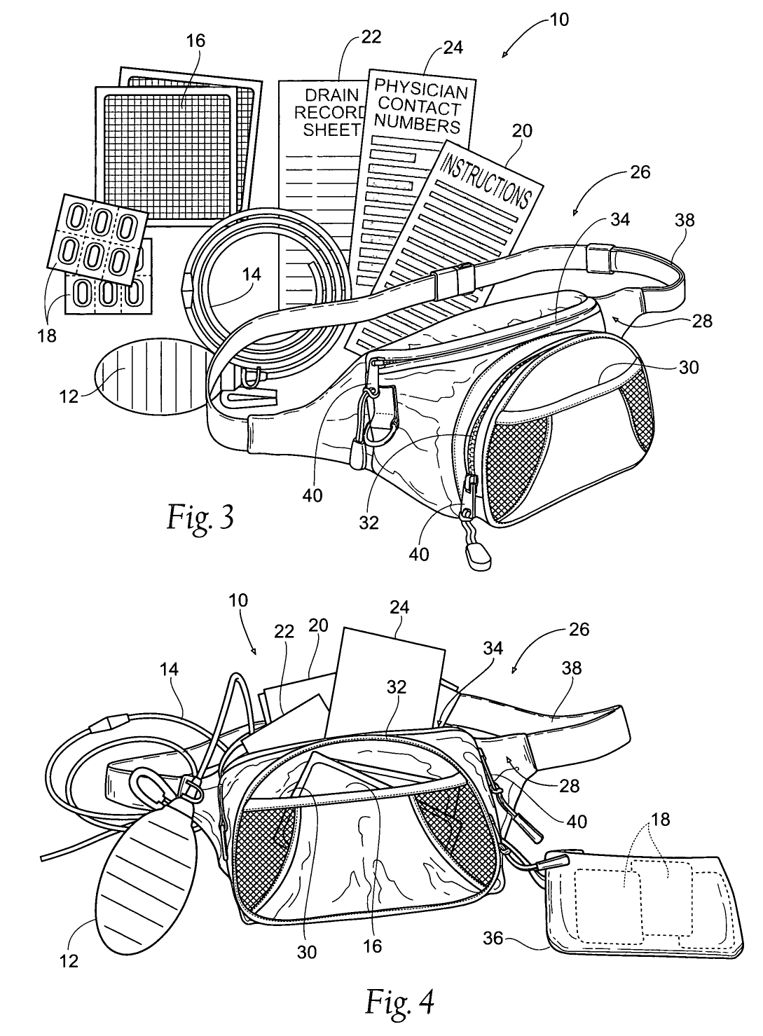

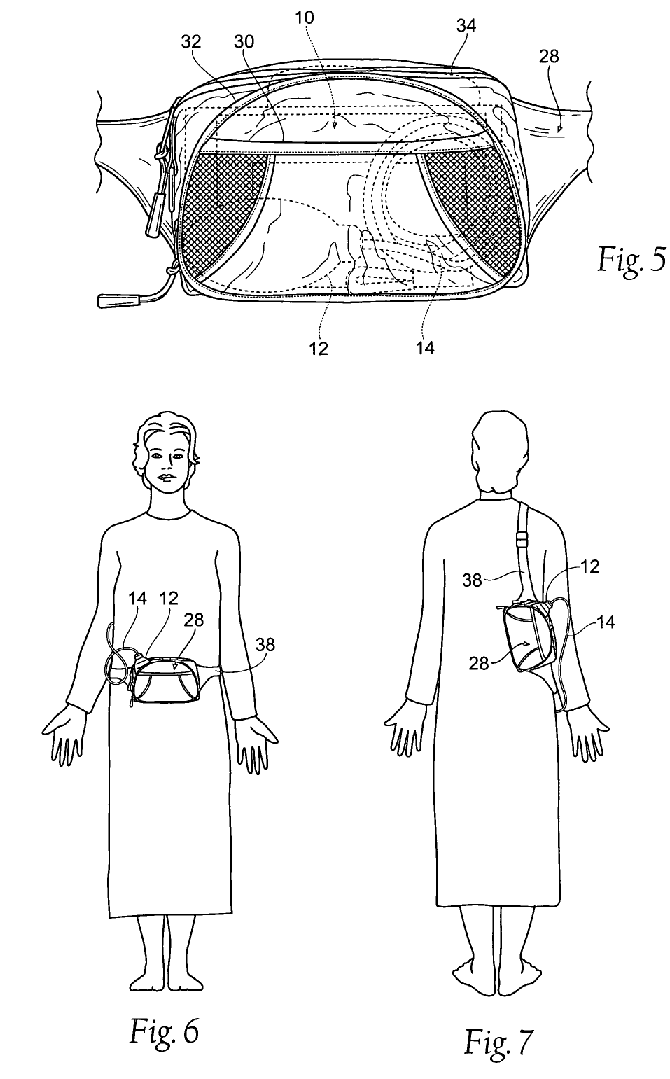

FIG. 3 shows a representative embodiment of a post-surgical accessory convenience kit 26, or PACK. The kit 26 includes all or some of the plurality of items 10 previously described and as shown in FIG. 2, which serve a patient's post-operative needs. As FIG. 3 shows, the kit 26 further includes a carrier or pouch 28. As shown in FIGS. 4 and 5, the pouch 28 includes one or more pockets 30; 32; 34 or attachable auxiliary cases 36 that are sized and configured to hold a number of the items 10 (such as shown in FIG. 2), which are directly related to the patient's post-operative needs. One or more auxiliary cases 36 can be attached by Velcro or carabineers on the external/outside surface of the pouch 28, as FIG. 4 shows. One or more of the pockets 30; 32; 34 and/or auxiliary cases 36 can be sized and configured to hold medication including pain medication, antibiotics and other medication, as well as dressings and dressing change materials. It should be understood that the kit of the present invention may include any combination of the items 10 listed above. It should be further understood that any additional item related to a patient's post-operative needs may be added to the kit.

The pouch 28 is desirably made of a material that is water and fluid resistant. The pouch includes a belt or strap 38 that makes it possible to secure the pouch 28 about the waist of a patient (see FIG. 6). The strap 38 could also be modified to allow the pouch 28 to be worn over the shoulder (see FIG. 7).

Some or all of the pockets 30; 32; 34 or attachable auxiliary cases 36 may include zippers or other suitable closure elements 40 (such as a slit or Velcro material). The closable pockets 30; 32; and 34 and auxiliary cases 36 can conveniently house surgical instructions, drain record sheets, and emergency physician and patient contact numbers, keeping these paper items protected from the elements and from loss. The pockets, when open, aid in the ease of removal of the contents out of the pocket.

At least one of the closable pockets 30; 34; 34 is desirably sized and configured to hold the drain suction bulb or reservoir 12 (which is coupled to the drain tubing 14 implanted in the patient), as well as other drain accessories. Placing the suction bulb 12 and the external length of tubing 14 within a pocket in the pouch 28 worn close to the incision and drain entry site can serve to relieve tension on the incision and drain entry site and avoid accidental pulling of the drain or suction bulb 12.

The pouch 28 can, if desired, be individualized with a patient's name, hospital or physician logos, or companies or practice names. After post-operative needs of the patient end, the pouch 28 can be cleaned, kept, and converted to everyday hiking or other non-medical uses.

The kit 26 may be supplied as a courtesy by caregivers to post-surgical patients, particularly those with surgical drains in place. For hospital based procedures, these would likely be covered by insurance. The kit 26 could also easily be incorporated into cosmetic surgeon's practices and given as a courtesy to patients post-operatively as a “bonus.”

Benefits of the kit 26 include convenience, comfort, accessibility, consolidation. The kit 26 makes it conveniently possible to have all desired or required post-operative items in one location, and the kit 26 goes where the patient goes. The kit 26 adds comfort by taking tension off of the incision and drain entry site and avoiding accidental pulling of the drain or suction bulb. The kit 26 is easily accessible and consolidates everything the patient needs with multiple pockets and locations for drain, dressings, and accessories. The kit 26 can be pre-packaged for each individual patient to hold Post-operative Instructions, Drainage Records, Contact numbers, Pain Medicine, Antibiotics, Other Medication, Antibiotic Ointment, Dressings, and, of course, the suction bulb for the drain.

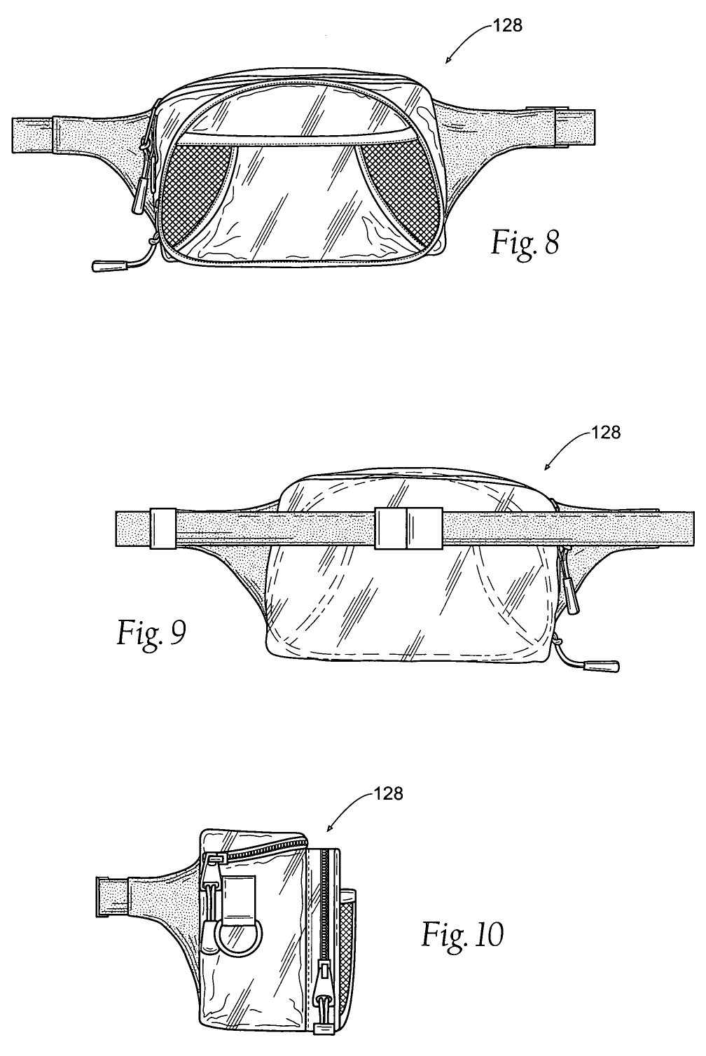

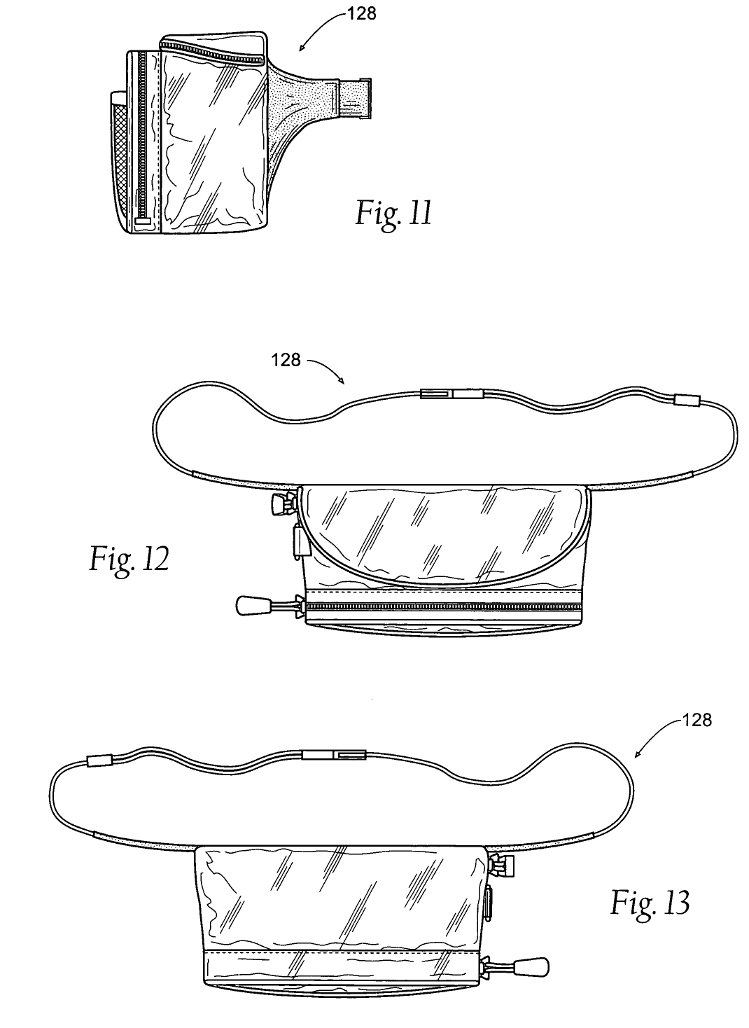

In an alternative embodiment of a pouch 128, as is shown in FIGS. 8 to 13, the pouch may be made of a see-through material. The see-through material may include, but is not limited to a clear plastic material, or a mesh metal or fabric. One or more of the items 10 shown in FIG. 2 may be placed within the pouch 128. In this manner the items 10 placed within the pouch 128 are visible from outside the pouch 128. Such a pouch 128 may be desirable in setting in which a patient must go through a security screening, such as at an airport. Often in a security screening an individual must have their bags sent through an x-ray machine or be hand searched. Removing the pouch 128 and sending the pouch 128 through an x-ray machine is not desirable to a patient with a drain. It may also be undesirable to have the pouch 128 hand searched. The see-through pouch allows security screeners to inspect the items 10 within the pouch 128 without removing the pouch 128 from the patient and without removing the items 10 from the pouch.

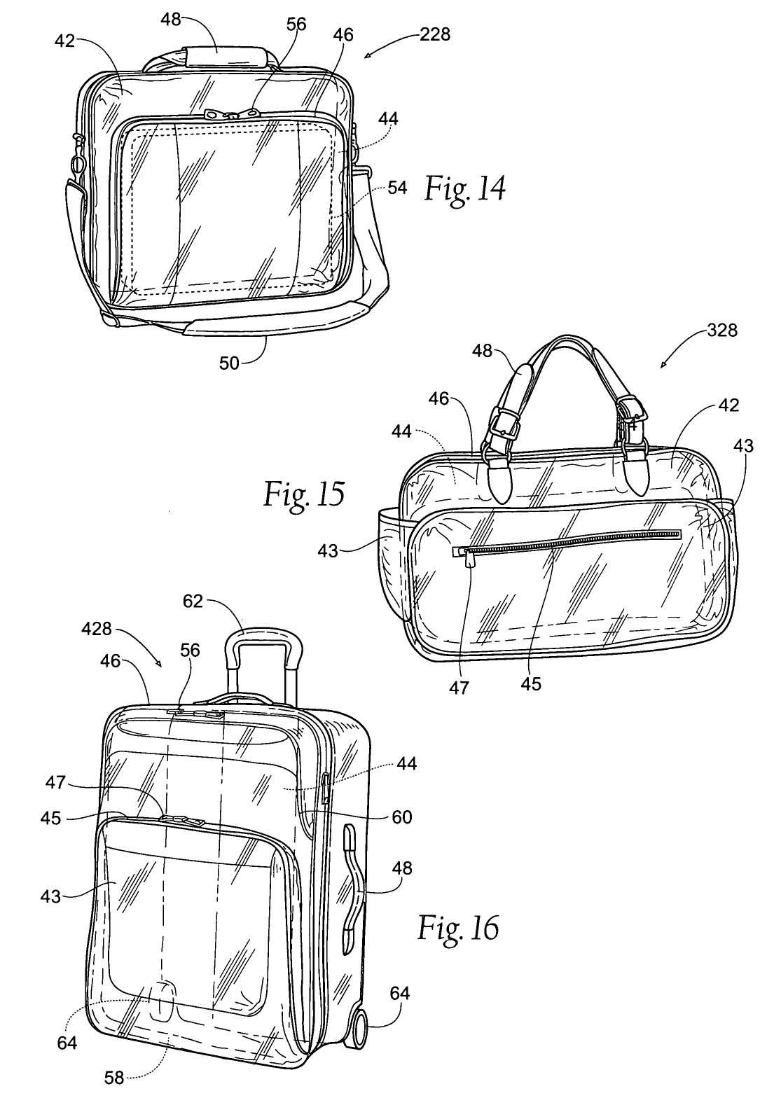

It is further contemplated that additional types of containers such as laptop cases or briefcases (see FIG. 14), carry-on bags (see FIG. 15), and suitcases (see FIG. 16) may be made of a see-through material. In this manner, the bag 228,328,428 may be inspected at a security checkpoint without sending the bag 228,328,428 through an x-ray machine or opening the bag 228,328,428 and searching the contents. As described above, the bag 228,328,428 may be made of any see-through material including, but not limited to a clear plastic material, or a mesh metal or fabric. It may be desirable to use such a bag 228,328,428 in any setting where security may be a concern and where it would be desirable to view the contents of a bag 228,328,428. For example, in a school, sports arena, airport, etc.

As shown in FIGS. 14 and 15, the bag 228,328 may include a body 42 having an interior volume 44. The body 42 may be made of a transparent material such that substantially the entire interior volume 44 is visible from outside the body 42. The bag 228,328 may include an opening 46 formed on the body to provide access to the interior volume 44. The bag 228,328 may include a handle 48 and/or a shoulder strap 50 coupled to the body 52. It is further contemplates that the interior volume 44 of the body 42 may be sized and configured to receive a laptop computer 54.

It is further contemplated that the interior volume 44 of the body 42 may be divided into a plurality of segregated storage compartments. It is contemplated that the segregated storage regions may be formed out of a transparent material. It is further contemplated that the bag 228,328 may include means for closure 56 of the interior volume 44. The means for closure 56 may include any means known in the art including, but not limited to a zipper or Velcro. The bag 228,328 may further include a flap which extends over the opening 46 to the interior volume 44. The bag 228,328 may further include at least one pocket 43 or storage compartment formed on one of the inside or the outside of the body 42. The pocket 43 may be formed of a transparent material. The pocket 43 preferably includes an opening 45 to access the interior of the pocket. The pocket may further include closure means 47, including but not limited to Velcro or a zipper, to close the pocket.

As seen in FIG. 16, the bag 428 may include a frame 58 having an interior volume 44 and a handle 48 coupled to the frame 58. The bag 428 may further include material 60 carried by the frame 58 to enclose the interior volume 44. The material 60 may be transparent such that substantially the entire interior volume 44 is visible from outside the frame 58.

The bag 428 may further include a telescoping handle 62. The bag 428 may further include at least one wheel 64 coupled to the frame 58. The bag 428 may further include an opening 46 on the bag 428 to provide access to the interior volume 44. It is further contemplated that the bag 428 may include means for closure 56 of the interior volume 44. The means for closure 56 may include any means known in the art including, but not limited to a zipper or Velcro.

The bag 428 may include a storage compartment sized and configured to receive a laptop computer. The storage compartment may be made of a transparent material. The bag 428 may further include at least one pocket 43 or storage compartment formed on one of the inside or the outside of the body 42. The pocket 43 may be formed of a transparent material. The pocket 43 preferably includes an opening 45 to access the interior of the pocket. The pocket 43 may further include closure means 47, including but not limited to Velcro or a zipper, to close the pocket.

The foregoing is considered as illustrative of the principles and technical features of the invention. Furthermore, since numerous modifications and changes will readily occur to those skilled in the art, it is not desired to limit the invention to the exact construction and operation shown and described. While the preferred embodiment has been described, the details may be changed without departing from the principles and technical features of the invention.

I/We claim:

1. A kit comprising:
a reservoir;
a length of tubing, the tubing having a first end sized and configured for coupling to the reservoir and a second end sized and configured for implanting into a drain site in a patient; and
a pouch.

2. A kit according to claim 1 further comprising at least one dressing change material.

3. A kit according to claim 1 further comprising at least one post-operative medication.

4. A kit according to claim 3 wherein said at least one post-operative medication includes at least one of a pain medication, an antibiotic, and an ointment.

5. A kit according to claim 1 further comprising at least one set of post-operative instructions.

6. A kit according to claim 1 further comprising at least one drain record.

7. A kit according to claim 1 further comprising at least one physician contact phone number.

8. A kit according to claim 1 wherein the pouch is made of a material that is water and fluid resistant.

9. A kit according to claim 8 wherein the material is clear.

10. A kit according to claim 1 wherein the pouch includes a strap.

11. A kit according to claim 10 wherein the strap is sized and configured to secure the pouch around the waist of a patient.

12. A kit according to claim 10 wherein the strap is sized and configured to wear the pouch over the shoulder of a patient.

13. A kit according to claim 1 wherein the pouch includes at least one pocket.

14. A kit according to claim 13 wherein at least one of the pockets of the pouch includes a closure element.

15. A kit according to claim 1 further comprising at least one auxiliary case attached to the pouch.

16. A method of providing post-operative materials to a patient, the method comprising:
providing a pouch, the pouch including a plurality of compartments;
providing a reservoir;
providing tubing;
providing at least one post-operative medication;
providing post-operative information;
providing at least one dressing change material;
arranging the reservoir, tubing, post-operative medication, post-operative information, and dressing change material within the compartments of the pouch; and
distributing the pouch to a patient.

17. A kit comprising:
a reservoir;
a length of tubing, the tubing having a first end sized and configured for coupling to the reservoir and a second end sized and configured for implanting into a drain site in a patient;
at least one dressing change material;
at least one post-operative medication;
a set of post-operative care instructions;
at least one physician contact phone number; and at least one drain record.

18. A kit according to claim 17 further comprising a pouch, said pouch having a plurality of compartment.

19. A kit according to claim 18 wherein each of said reservoir, dressing change material, at least one post-operative medication, set of post-operative care instruction, at least one physician contact phone number, and at least one drain record are contained within one of the compartments of the pouch.

20. A luggage article comprising
a frame having an interior volume;
a handle coupled to the frame; and
material carried by the frame to enclose the interior volume, the material being transparent such that substantially the entire interior volume is visible from outside the frame.

21. A luggage article according to claim 20 wherein said handle is a telescoping handle.

22. A luggage article according to claim 20 further comprising at least one wheel coupled to the frame.

23. A luggage article comprising:
a body having an interior volume, the body being made of a transparent material such that substantially the entire interior volume is visible from outside the body;
an opening on said body, the opening providing access to said interior volume; and
at least one of a handle and strap coupled to the body.

24. A luggage article according to claim 23 wherein said interior volume is sized and configured to receive a laptop computer.
